(19) United States
(12) Patent Application Publication
(10) Pub. No.: US 2019/0351069 A1
(43) Pub. Date: Nov. 21, 2019

(54) Coating of Nanoparticle Surfaces with Cyclopeptides for Improving Delivery of Agents Via the Oral Route

(71) Applicant: Ruprecht-Karls-Universität Heidelberg, Heidelberg (DE)

(72) Inventors: Philipp Uhl, Heidelberg (DE); Walter Mier, Bensheim (DE); Uwe Haberkorn, Schwetzingen (DE); Max Sauter, Heidelberg (DE)

(21) Appl. No.: 16/329,534

(22) PCT Filed: Sep. 19, 2017
(86) PCT No.: PCT/EP2017/001119

§ 371 Date: Feb. 28, 2019

(30) Foreign Application Priority Data
Oct. 5, 2016 (EP) 16002139.0

(51) Int. Cl. A61K 47/69; A61K 9/19; A61K 9/00; A61K 9/48; A61K 38/12; A61K 31/704; A61K 38/28; A61K 38/26

(52) U.S. Cl. CPC A61K 47/6937; A61K 9/19; A61K 9/006; A61K 38/26; A61K 38/12; A61K 31/704; A61K 38/28; A61K 9/4808

(57) ABSTRACT

The present invention relates to a surface-coated nanoparticle comprising biodegradable polymer chains and cyclopeptides, wherein the cyclopeptides are covalently attached to the biodegradable polymer chains forming the nanoparticle, thereby coating the surface of the nanoparticle. The present invention further relates to the use of said surface-coated nanoparticle as a capsule for an agent for improving the oral delivery thereof. In another aspect, the present invention relates to the surface-coated nanoparticle for use in the treatment or prevention of a disorder or disease in a patient, wherein the treatment or prevention is achieved by mucosal uptake of the surface-coated nanoparticle via the oral route.

The present invention relates to a surface-coated nanoparticle and to the use thereof as a capsule for an agent. In another aspect, the present invention relates to the surface-coated nanoparticle for use in the treatment or prevention of a disorder or disease in a patient, wherein the treatment or prevention is achieved by mucosal uptake of the surface-coated nanoparticle via the oral route.

Oral drug delivery is considered as the most advantageous way of application, in particular for the treatment of chronic diseases which demand long-term and repeated drug administration. The oral route offers high drug safety and is widely accepted among patients due to its convenience. In addition, non-sterility of oral drug forms reduces costs in production, storage and distribution, which could contribute to health care improvement in third world countries. It is estimated that 90% of all marketed drug formulations are for oral use.

However, the oral delivery of many promising drugs is limited due to their instability in the gastrointestinal tract and due to their low mucosa penetration. In particular, macromolecular drugs show a very poor stability under the acidic conditions in the stomach after oral administration as well as a poor uptake across the gastrointestinal barrier. Up to now, macromolecular drugs thus have to be administered either subcutaneously or intravenously, causing high medical costs and low patient compliance.

To overcome the afore-mentioned problems, different approaches which are directed to the improvement of the bioavailability of orally administered drugs have been tested in the past years, including nano- or microemulsions, polymeric micelles, and hydrogel microparticles. However, said approaches suffer from both a rapid removal by the mucus and a poor mucosal uptake. Even pH-responsive complexation gels could not provide results sufficient for clinical use due to low mucosa penetration. Besides, liposomes have been examined for improving the oral delivery of drugs, but conventional liposomal formulations have not been very convincing due to their instability in the gastrointestinal tract. In this context, a significant improvement in liposomes can be achieved by the combination of conventional phospholipids and so-called tetraether lipids. Recent studies have shown that these tetraether lipids can improve the liposomal stability in the gastrointestinal tract and can mediate the mucosal uptake.

However, the above-mentioned approach using liposomes stabilized with tetraether lipids is not suitable for an industrial production, since said tetraether lipids can only be isolated from specific bacteria such as archaea, e.g. the extremophilic archaeon Sulfolobus acidocaldarius. Accordingly, the availability of such tetraether lipids is limited to the laboratory scale.

Accordingly, there is a need for alternative and new approaches which aim at the improvement of oral delivery of drugs, in particular of macromolecular drugs.

In view of the above, the technical problem underlying the present invention is the provision of means for the delivery of agents, e.g. drugs, via the oral route, having enhanced mucosal uptake and bioavailability.

The solution to the above technical problem is achieved by providing the embodiments characterized in the claims.

In particular, in a first aspect, the present invention relates to a surface-coated nanoparticle, said surface-coated nanoparticle comprising:
a nanoparticle comprising biodegradable polymer chains terminated with a functional group for being functionalized with a cyclopeptide, and
cyclopeptides covalently attached to the biodegradable polymer chains via the functional group thereof, thereby coating the surface of the nanoparticle.

Using the above-defined surface-coated nanoparticle as a capsule for an agent, in particular for a macromolecular agent, not only allows enhancing the mucosal uptake of the encapsulated agent via the oral route, but also allows safely transporting the latter through the acidic milieu of the gastrointestinal tract. In contrast to the abovementioned liposomal approach, the present invention is readily applicable in an industrial scale, since all starting materials are either commercially available or synthetically accessible.

According to the present invention, the above-defined surface-coated nanoparticle comprises biodegradable polymer chains terminated with a functional group for being functionalized with a cyclopeptide. As used herein, the term “biodegradable polymer” relates to any polymer that is capable of decaying through the action of bacteria, fungi, or other biological means. Preferably, the degradation of the biodegradable polymer chains takes place within the cells after mucosal uptake of the surface-coated nanoparticle via the oral route. Further, the term “chain” relates to a polymeric molecule of the biodegradable polymer, comprising the respective monomers.

In one specific embodiment of the above-defined surface-coated nanoparticle, the biodegradable polymer is polylactide. Accordingly, in this specific embodiment, the nanoparticle comprises polylactide chains terminated with a functional group for being functionalized with a cyclopeptide. Polylactide is a biodegradable material which is used in various medical applications. In line with the above definition, the term “polylactide chain” relates to a polymeric molecule comprising lactic acid monomers. In this context, the terms “polylactide” and “polylactic acid”, abbreviated as PLA, have the same meaning.

The average molecular weight of the modified biodegradable polymer chains is not specifically limited according to the present invention. Herein, the terms “biodegradable polymer chain terminated with a functional group” and “modified biodegradable polymer chain” are used synonymously. In one embodiment of the present invention, the biodegradable polymer chains terminated with a functional group for being functionalized with a cyclopeptide have an average molecular weight of from 1,000 to 10,000 g/mol, preferably of from 2,000 to 8,000 g/mol, more preferably of from 3,000 to 7,000 g/mol, and particularly preferably of from 4,000 to 6,000 g/mol. For example, the surface-coated nanoparticle according to the present invention may comprise modified biodegradable polymer chains having an average molecular weight of 5,000 g/mol.

It is well known in the art that biodegradable polymer chains can form nanoparticles by aggregation, mediated by non-covalent interactions between the biodegradable polymer chains. The same applies to biodegradable polymer chains that are modified with a functional group at one terminus. Herein, the term “nanoparticle” is defined as a particle having an average particle size of not more than 500 nm, preferably of not more than 300 nm, more preferably of not more than 200 nm, and particularly preferably of not more than 150 nm, in all three dimensions.

According to the present invention, the stereoregularity of the modified biodegradable polymer chains forming the nanoparticle is not limited. The modified biodegradable polymer chains may be isotactic, syndiotactic, heterotactic or atactic. In line with this, in case the biodegradable polymer is polylactide, the modified polylactide chains may comprise monomers of pure (S)-lactic acid or monomers of pure (R)-lactic acid, and may also be formed of a racemic mixture of both enantiomers or of mixtures with different molar ratios thereof. Herein, any arbitrary tacticity of the modified polylactide chains is included.

Besides the modified biodegradable polymer chains, the surface-coated nanoparticle as defined above optionally comprises unmodified biodegradable polymer chains, i.e. biodegradable polymer chains which are not terminated with a functional group for being functionalized with a cyclopeptide. In a specific embodiment of the present invention, both modified and unmodified biodegradable polymer chains form the nanoparticle. All relevant limitations and definitions provided for the modified biodegradable polymer chains according to the present invention apply to the unmodified biodegradable polymer chains in an analogous manner.

In the above-defined surface-coated nanoparticle, the modified biodegradable polymer chains forming the nanoparticle are functionalized with cyclopeptides, also known as cyclic peptides, thereby coating the surface of the nanoparticle. According to the present invention, the degree of functionalization is not limited. In a specific embodiment, the degree of functionalization is at least 0.1%, preferably at least 0.5%, more preferably at least 1%, and particularly preferably at least 2%. Furthermore, in another specific embodiment, the degree of functionalization is at most 10%, preferably ably at most 8%, more preferably at most 6%, and particularly preferably at most 5%. Herein, the degree of functionalization means the ratio of the modified biodegradable polymer chains which are functionalized with a cyclopeptide to the total of modified and unmodified biodegradable polymer chains forming the nanoparticle.

As used herein, the term “functionalization” means the covalent attachment of a cyclopeptide to a modified biodegradable polymer chain. According to the present invention, the functional group of the modified biodegradable polymer chains is not particularly limited as long as it is capable of being functionalized with a cyclopeptide. In this respect, suitable functional groups are, for example, amino groups optionally substituted with one or more hydrocarbon groups having 1 to 6 carbon atoms, N-hydroxysuccinimide, maleimide, and others. In a specific embodiment of the above-defined surface-coated nanoparticle, the biodegradable polymer chains are terminated with a maleimide moiety. For example, maleimide-modified polylactide chains having a molecular weight of 5,000 g/mol are commercially available and can, for example, be purchased from Sigma Aldrich.

In the surface-coated nanoparticle as defined above, the cyclopeptides are covalently attached to the biodegradable polymer chains via the functional group thereof, thereby coating the surface of the nanoparticle. Accordingly, the cyclopeptides need to have a functional group for functionalizing the modified biodegradable polymer chains, i.e. for being covalently attached thereto. For example, the cyclopeptides may have a functional group such as amino, thiol, hydroxyl, maleimide etc. However, the cyclopeptides according to the present invention are not limited to any specific functional group for covalent attachment to the modified biodegradable polymer chains. In particular, the modified biodegradable polymer chains and the cyclopeptides in the above-defined surface-coated nanoparticle may be functionalized in any way, as long as the covalent attachment thereof is ensured.

In a specific embodiment of the present invention, the cyclopeptides have at least one hydroxyl and/or thiol group. In this case, the cyclopeptides are covalently attached to the modified biodegradable polymer chains via the reaction of the functional group of the modified biodegradable polymer chains with the at least one hydroxyl and/or thiol group. For example, in case the biodegradable polymer chains are terminated with a maleimide moiety, the at least one hydroxyl and/or thiol group of the cyclopeptides attaches to the double bond of the maleimide moiety of the modified biodegradable polymer chains by an addition reaction.

Accordingly, in the above specific embodiment, the cyclopeptides used for functionalizing the modified biodegradable polymer chains comprise at least one amino acid having a hydroxyl and/or thiol group, such as tyrosine, threonine, serine or cysteine. In a further specific embodiment of the above-defined surface-coated nanoparticle, the cyclopeptides comprise at least one cysteine moiety. In this case, the cyclopeptides are covalently attached to the modified biodegradable polymer chains via their at least one thiol group.

In order to prevent the cyclopeptides from acting as crosslinkers between two or more modified biodegradable polymer chains, it is preferred that the cyclopeptides used for functionalizing the modified biodegradable polymer chains comprise at most one hydroxyl group or at most one thiol group. Preferably, at most one amino acid selected from the group consisting of tyrosine, threonine, serine and cysteine is thus included in the cyclopeptides according to the present invention.

In the above-defined surface-coated nanoparticle, the cyclopeptides covalently attached to the modified biodegradable polymer chains are preferably capable of penetrating cells. In general, cell-penetrating peptides (CPPs) are able to penetrate cell membranes and translocate different cargoes into the cells, wherein the potential cargoes can range from small molecules to proteins.

Various structures of cell-penetrating peptides are known in the art, including linear as well as cyclic forms thereof, e.g. penetratin (SEQ ID NO: 1; RQIKIWFQNRRMKWKK), derived from Drosophila melanogaster, TAT (transactivator of transcription)-peptide (SEQ ID NO: 2; CGRKKKRRQRRRPPQC), derived from HIV-1, MAP (model amphiphatic peptide) (SEQ ID NO: 3; GALFLGFLGAAGSTMGAWSQPKSKRKV), which is an artificial peptide, R9 (SEQ ID NO: 4; RRRRRRRRR), which is an artificial peptide, pVEC (SEQ ID NO: 5; LLIILRRRIRKQAHAHSK-amide), which is a CPP derived from murine vascular endothelial cadherin, transportan (SEQ ID NO: 6; GWTLNSAGYLLGKINLKALAALAKISIL-amide), which is derived from the human neuropeptide galanin, and MPG (SEQ ID NO: 7; GALFLGFLGAAGSTMGAWSQPKSKRKV), which is derived from HIV, combinations thereof, and dimers thereof.

The cyclopeptides of the surface-coated nanoparticle according to the present invention are, however, not limited to any of the cyclic forms of the above cell-penetrating peptides or to any derivatives thereof. As mentioned above, the only requirement is the presence of a functional group for covalent attachment to the modified biodegradable polymer chains.

As used herein, the term “cell” relates to the epithelial cells of the mucosa, and the term “mucosal uptake” means the penetration of the epithelial cells of the mucosa by the surface-coated nanoparticle. According to the present invention, mucosal uptake refers to the uptake of the surface-coated nanoparticle by the mucosa of the nostrils, the lips of the mouth, the eyelids, the ears, the trachea, the stomach, the gastrointestinal tract, preferably the small intestine, the duodenum, the jejunum, the ileum, or the large intestine, the cecum, the colon, the rectum, the anal canal, the anus, and the genital area.

In contrast to linear CPPs, cell penetrating cyclopeptides are less susceptible to hydrolysis by peptidases, i.e. they have been shown to be enzymatically more stable. Accordingly, in the surface-coated nanoparticle as defined above, cyclopeptides, preferably cell-penetrating cyclopeptides, are used for functionalizing the modified biodegradable polymer chains. In case the cyclopeptides of the surface-coated nanoparticle as defined above are cell-penetrating, the mucosal uptake of the surface-coated nanoparticle via the oral route is improved.

The preparation of the nanoparticle comprising the modified biodegradable polymer chains, and optionally the unmodified biodegradable polymer chains, as well as its functionalization with the cyclopeptides are not particularly limited according to the present invention. For example, a double emulsion technique known in the art, which makes use of a surfactant, may be applied for this purpose.

When a surfactant is present during the preparation of the surface-coated nanoparticle, the characteristics thereof can be controlled. Specifically, the nanoparticle size, i.e. the number of modified, and if present unmodified, biodegradable polymer chains forming the nanoparticle can be adjusted by the respective surfactant, in particular, by its value of hydrophilic-lipophilic balance (HLB). Besides, the surfactant helps to prevent the agglomeration of the surface-coated nanoparticles. In this context, it is assumed that the surfactant shell formed around a surface-coated nanoparticle undergoes repulsive interactions with the surfactant shell formed around an adjacent surface-coated nanoparticle. According to the present invention, the surfactant is merely required for the preparation of the surface-coated nanoparticle. Afterwards, it may remain or it may be removed without any loss of stability.

For example, HLB values of the surfactant for preparing the surface-coated nanoparticle are from 6 to 18, preferably from 8 to 16, more preferably from 9 to 15, and particularly preferably from 10 to 14. When the HLB value of the surfactant falls within the range of from 6 to 18, nanoparticle sizes between 50 and 500 nm can be obtained. In particular, when using a surfactant with a HLB value of from 10 to 14, the size of the surface-coated nanoparticle is between 50 and 200 nm. As used herein, the terms “size” and “nanoparticle size” mean the average size of the above-defined surface-coated nanoparticle comprising an agent encapsulated therein, with the latter being described in more detail below. When the nanoparticle size falls within 50 and 500 nm, preferably within 60 and 400 nm, more preferably within 80 and 300 nm, and particularly preferably within 100 and 200 nm, the mucosal uptake of the surface-coated nanoparticle according to the present invention is significantly enhanced compared to smaller or larger nanoparticle sizes, thereby improving the oral delivery of the encapsulated agent. In one embodiment, polyvinyl alcohol (PVA) with a HLB value of 18 is used as the surfactant for preparing the surface-coated nanoparticle. However, the surfactant is preferably selected from the group consisting of Tween 85 and Cremophor® EL, which have a HLB value of 11 and 12, respectively.

The cyclopeptides for functionalizing the modified biodegradable polymer chains in the above-defined surface-coated nanoparticle can be produced by any suitable method known in the art, e.g. by a solid-phase synthesis using suitable protecting groups.

As mentioned above, the cyclopeptides are covalently attached to the modified biodegradable polymer chains of the nanoparticle, thereby functionalizing the nanoparticle and coating the surface thereof. According to the present invention, a bifunctional linker may be attached between the cyclopeptides and the modified biodegradable polymer chains. The bifunctional linker may be present or may be absent, as required. Suitable bifunctional linkers for linking the cyclopeptides and the modified polylactide chains are known in the art. According to the present invention, the bifunctional linker is not particularly limited.

In a specific embodiment of the surface-coated nanoparticle as defined above, it is preferred that the linker for linking the cyclopeptides and the modified biodegradable polymer chains is a bifunctional polyethylene glycol (PEG) linker. When covalently attaching the cyclopeptides to the modified biodegradable polymer chains via a bifunctional PEG linker, a prolonged mucosa adhesion can be achieved by such PEGylation, and the surface-coated nanoparticle is less prone to a rapid removal by the mucus. The reason for this enhanced mucoadhesivity may be seen in the attractive interaction of the PEG side chains with the mucin network of the mucosa.

In one embodiment, the bifunctional polyethylene glycol linker has between 1 and 200 PEG moieties, preferably between 2 and 150 PEG moieties, more preferably between 4 and 100 PEG moieties, and particularly preferably between 8 and 50 PEG moieties.

Without limitation, the bifunctional PEG linker may be first attached to the cyclopeptide. Then, in a second step, the obtained adduct may react with the functional group of the biodegradable polymer chain. For example, the bifunctional PEG linker may have a maleimide moiety for being attached to the cyclopeptide, and may have an N-hydroxysuccinimidyl ester group for being attached to the biodegradable polymer chain which in turn may be terminated with an amino group. However, it is clear that depending on the functionalities of the respective reactants, it is also possible that the bifunctional PEG linker first reacts with the functional group of the biodegradable polymer chain before being attached to the cyclopeptide.

The molecular weight and the size of the cyclopeptides according to the present invention are not specifically limited. However, it is preferred that the total number of amino acids forming a cyclopeptide molecule is equal to or less than 30, more preferred equal to or less than 25, and particularly preferred equal to or less than 20.

As used herein, the term “cyclopeptide” is not to be construed as a peptide having one ring system only, i.e. the present invention is not limited to monocyclic peptides. Accordingly, the present invention also relates to cyclopeptides, wherein two or more ring systems are covalently linked to each other. Furthermore, the cyclopeptides of the above-defined surface-coated nanoparticle may also comprise amino acids which are not part of the ring system. Thus, peptide side chains may be present in the cyclopeptides. Preferably, the cyclopeptides used for functionalizing the modified biodegradable polymer chains are monocyclic peptides, and more preferably monocyclic peptides having no peptide side chains.

In a preferred embodiment of the present invention, the cyclopeptides are positively charged. Thereby, the mucosal uptake of the surface-coated nanoparticle is enhanced. For example, the cyclopeptides of the surface-coated nanoparticle as defined above may comprise mostly lysine and/or arginine moieties, which have isoelectric points of around 9.5 and 11, respectively. Due to their additional amino group, these two amino acids are positively charged under neutral and even under weakly basic conditions. Accordingly, a cyclopeptide mostly comprising moieties of said two specific amino acids is positively charged under neutral and weakly basic conditions as well. Herein, the term “mostly comprising” means that at least 50%, preferably at least 60%, more preferably at least 70%, and particularly preferably at least 80% of the amino acids forming a cyclopeptide molecule are lysine and/or arginine moieties. Thereby, it is ensured that the cyclopeptides have a positive charge under neutral and weakly basic conditions, i.e. their isoelectric point is higher than 7. Therefore, in a specific embodiment of the present invention, the isoelectric point of the cyclopeptides of the above-defined surface-coated nanoparticle is higher than 7.0, preferably higher than 7.5, more preferably higher than 8.0, and particularly preferably higher than 8.5. In this context, the isoelectric point of the cyclopeptide is the arithmetic mean of the isoelectric points of the amino acids forming the cyclopeptide.

In a specific embodiment of the present invention, the cyclopeptides of the abovedefined surface-coated nanoparticle comprise between 2 to 19, preferably between 3 to 16, more preferably between 4 to 14, and particularly preferably between 6 to 12 arginine moieties as well as one moiety selected from the group consisting of tyrosine, threonine, serine and cysteine. For example, the cyclopeptides used for functionalizing the modified biodegradable polymer chains comprise nine arginine moieties and one cysteine moiety in the ring system, and are referred to as a cyclic cysteine R9 derivative (SEQ ID NO: 8; RRRRRRRRRC).

According to the present invention, the amino acids forming the cyclopeptides of the surface-coated nanoparticle are not limited to proteinogenic amino acids. Herein, the amino acids may be selected from any amino acids known in the art, and may include the respective D-enantiomer, L-enantiomer, or any mixture thereof. Herein, the amino acids may be further functionalized so as to covalently attach to the modified biodegradable polymer chains.

In a further aspect, the present invention relates to the use of the above-defined surface-coated nanoparticle as a capsule for an agent. According to the present invention, the term “capsule” and any other term derived therefrom such as “encapsulating” mean that the agent is embedded in the inside of the nanoparticle, i.e. the agent is surrounded by the modified, and if present, by the unmodified biodegradable polymer chains, with some of the modified biodegradable polymer chains being functionalized with the cyclopeptides. In the above-defined surface-coated nanoparticle, the encapsulated agent is not covalently attached to any part thereof.

The surface-coated nanoparticle according to the present invention may be regarded as a delivery system for an agent. With the agent being encapsulated in the surface-coated nanoparticle, the mucosal uptake of the agent via the oral route is significantly enhanced, and it is safely transported through the acidic milieu of the gastrointestinal tract.

Accordingly, in another embodiment of the above-defined surface-coated nanoparticle, the surface-coated nanoparticle further comprises an encapsulated agent. In this context, the term “agent” relates both to a therapeutic agent and to a diagnostic agent, i.e. to a drug, but is not limited thereto. Herein, the term “agent” also relates to any excipient or additive, which is pharmaceutically acceptable. In this respect, the excipient may be e.g. a preservative like an antioxidant such as ascorbic acid, but the present invention is not limited thereto. According to the present invention, the agent encapsulated in the surface-coated nanoparticle may be a macromolecular agent. In one embodiment of the surface-coated nanoparticle as defined above, the agent is selected from the group consisting of peptides, proteins, antibodies, and combinations thereof. In another embodiment of the surface-coated nanoparticle, the agent is selected from the group consisting of nucleic acids, synthetic conjugates and small molecules.

As used herein, the term “macromolecular agent” relates to an agent which has a molecular weight of at least 500 g/mol, for example, between 20,000 and 200,000 g/mol (FIG. 1). In a specific embodiment of the present invention, the macromolecular agent has a molecular weight of from 1,000 to 10,000 g/mol. Furthermore, the term “small molecule” relates to an agent having a molecular weight of at most 500 g/mol, preferably of at most 400 g/mol, and more preferably of at most 300 g/mol.

According to the present invention, the agent is not particularly limited, and is preferably a therapeutic and/or diagnostic agent for which an oral delivery by mucosal uptake might be interesting, including vaccines, preferably oral vaccines. Herein, the term “agent” may be used in a singular form, but it is not limited to only one specific therapeutic agent or to only one specific diagnostic agent as well as not limited to only one specific excipient or to only one specific additive. In addition, the term “agent” is not to be construed as referring to only one molecule thereof. Accordingly, in the above-defined surface-coated nanoparticle, numerous molecules of one or more such agents may be encapsulated. Therefore, the singular form of the term “agent” as used herein is by no means limiting.

In one specific embodiment, the surface-coated nanoparticle according to the present invention comprises the drug Myr-HBVpreS/2-48 (Myrcludex B) as an agent encapsulated therein. Myrcludex B is a novel lipopeptide and the first in a new class of hepatitis B drugs, which has been shown promising in the prevention or treatment of hepatic disorders or diseases. Previous findings have shown that as a virus entry inhibitor which specifically accumulates in the liver, Myrcludex B can block hepatitis B virus (HBV) entry in vitro and in vivo.

In detail, Myrcludex B is a linear peptide comprising 47 amino acids corresponding to amino acids 2 to 48 of the hepatitis B virus preS protein (SEQ ID NO: 9; GQNLSTSNPLGFFPDHQLDPAFRANTANPDWDFNPNKDTWPDANKVG) with a myristoylation on the N-terminus, having a molecular weight of 5366 g/mol. It is an investigational drug for hepatitis B treatment. However, as a macromolecular agent, Myrcludex B per se shows only poor oral bioavailability (<1%), so that only subcutaneous application is so far possible, resulting in low patient compliance and high medical costs. However, when encapsulated in the surface-coated nanoparticle according to the present invention, the oral bioavailability and hepatic delivery of Myrcludex B can be significantly increased. Therefore, the surface-coated nanoparticle as defined above can be for use in the prevention and/or treatment of hepatitis B.

Moreover, in the present invention, Myrcludex B has been identified as showing pronounced hepatotropism, i.e. exhibiting a high selective accumulation in the liver tissue. Therefore, the surface-coated nanoparticle according to the present invention can be used as an orally administered drug delivery system exhibiting a high hepatic targeting efficacy.

In another specific embodiment, the surface-coated nanoparticle according to the present invention comprises the drug Liraglutide as an agent encapsulated therein. Liraglutide is an analogue of the incretin glucagon-like peptide-1 (GLP-1) indicated for the treatment of type 2 diabetes mellitus and adiposity—two diseases with increasing occurrence in modern western civilizations. Up to date, Liraglutide has to be administered daily via subcutaneous injection.

According to the present invention, the encapsulation of the agent in the surface-coated nanoparticle can be achieved by any appropriate means known in the art. For example, the modified biodegradable polymer chains, and optionally the unmodified biodegradable polymer chains, may be dispersed together with the agent in the presence of a surfactant, resulting in the encapsulation of the agent by the in situ formed nanoparticle. In a next step, the functionalization of the modified biodegradable polymer chains of the nanoparticle comprising the encapsulated agent can be carried out by adding the cyclopeptides, resulting in the coating of the nanoparticle surface by the covalent attachment of the cyclopeptides to the modified biodegradable polymer chains.

In the above-defined surface-coated nanoparticle, the functionalization of the modified biodegradable polymer chains is not limited to only one specific cyclopeptide. According to the present invention, more than one specific cyclopeptide may be used for this purpose.

The specific embodiments and definitions of the agent according to the present invention relate both to the above-defined surface-coated nanoparticle comprising said agent encapsulated therein as well as to the above-defined use of the surface-coated nanoparticle as a capsule for said agent.

In another embodiment, the above-defined surface-coated nanoparticle is in a freeze-dried state in the presence of a lyoprotectant, e.g. disaccharides such as sucrose, which allows the long term storage of the surface-coated nanoparticle. For example, freeze-drying can be accomplished using 500 to 1000 mM, preferably 600 to 900 mM, and more preferably 700 to 800 mM sucrose. Thereby, the size of the surface-coated nanoparticle is maintained even after storing for several months, thus not leading to a significant change in the mucosal uptake thereof.

In another aspect, the present invention relates to a composition comprising the surface-coated nanoparticle as defined above, which may also be referred to as a pharmaceutical composition.

In a further aspect, the present invention relates to a capsule comprising the above-defined surface-coated nanoparticle or the above-defined composition after freeze-drying, i.e. the nanoparticles themselves are encapsulated. The capsule for encapsulating the nanoparticle may be seen as a packaging for the nanoparticle. In a preferred embodiment of the present invention, the capsule is gastro-resistant, thereby ensuring the safe transport of the surface-coated nanoparticle and the agent encapsulated therein through the acidic milieu of the gastrointestinal tract. Accordingly, the agent which is encapsulated by the nanoparticle is further protected by the capsule which encapsulates the nanoparticle. Preferably, the capsule for encapsulating the nanoparticle is a hard-shelled capsule which is typically made of gelatin, without being limited thereto though.

In another aspect, the present invention relates to a tablet comprising the above-defined surface-coated nanoparticle or the above-defined composition after freeze drying, i.e. the nanoparticles are pressed into a tablet. Preferably, the tablet is covered with a gastro-resistant coating to ensure the safe transport of the surface-coated nanoparticle and the agent encapsulated therein through the acidic milieu of the gastrointestinal tract. Suitable gastro-resistant coatings which may be applied herein are known in the art.

All relevant limitations and definitions provided for the surface-coated nanoparticle according to the present invention, including the nature of the agent, apply to the above-defined composition and to the above-defined capsule as well as to the above-defined tablet in an analogous manner. In particular, the composition and the capsule as well as the tablet according to the present invention may comprise the surface-coated nanoparticle with or without an agent encapsulated therein. In particular, the therapeutic and diagnostic agents as well as the hepatic disorders and diseases are as defined above.

In another aspect, the present invention relates to the above-defined surface-coated nanoparticle, to the above-defined composition, and to the above-defined capsule as well as to the above-defined tablet for use in a method of treatment of the human or animal body.

In a related aspect, the present invention also relates to the use of the above-defined surface-coated nanoparticle, to the use of the above-defined composition, and to the use of the above-defined capsule as well as to the use of the above-defined tablet in a method of treatment of the human or animal body.

In yet another aspect, the present invention relates to the above-defined surface-coated nanoparticle, to the above-defined composition, and to the above-defined capsule as well as to the above-defined tablet for use in the treatment or prevention of a disorder or disease in a patient, wherein the treatment or prevention is achieved by mucosal uptake of the surface-coated nanoparticle via the oral route.

In a related aspect, the present invention also relates to the use of the above-defined surface-coated nanoparticle, to the use of the above-defined composition, and to the use of the above-defined capsule as well as to the use of the above-defined tablet in the treatment or prevention of a disorder or disease in a patient, wherein the treatment or prevention is achieved by mucosal uptake of the surface-coated nanoparticle via the oral route.

According to the present invention, the terms “treatment” and “prevention” are not limited to the treatment and prevention of a certain disorder or disease. For example, the agent encapsulated in the surface-coated nanoparticle may be for the treatment or prevention of sepsis, diabetes, rheumatism, acromegaly, all kinds of hepatitis, all kinds of cancer, and anemia. Furthermore, the term “prevention” also includes vaccination, preferably oral vaccination. A specific embodiment of the present invention relates to the treatment and prevention of hepatic disorders or diseases, in particular the hepatitis B virus. Furthermore, the term “patient” means both humans and animals, preferably vertebrates, more preferably mammals.

### THE FIGURES SHOW

FIG. 1: Peptide drugs and other biologicals show poor oral availability with increasing size. Until today, various drugs need to be applied either subcutaneously or intravenously.

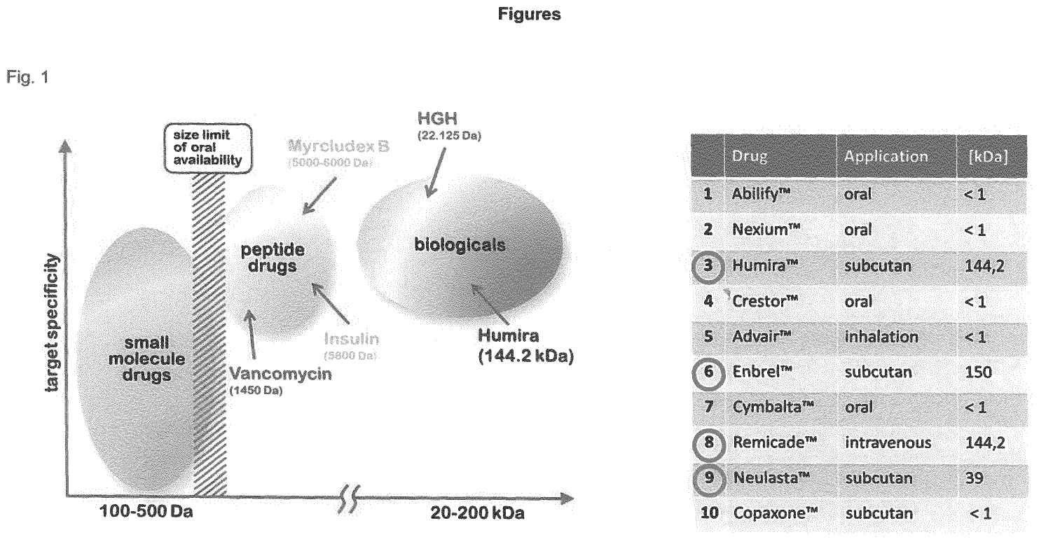

FIG. 2: Principle illustrating the functionalization of a maleimide-modified polylactide chain with the cyclic cysteine R9 derivative (SEQ ID NO: 8; RRRRRRRRRC). “PLA” represents polylactide, “R” represents arginine, and “C-SH” represents cysteine with its thiol group.

FIG. 3: The size and the polydispersity index (PDI) of the surface-coated nanoparticles are plotted versus various surfactants (n≥5). The size without drug for the Span 85 formulation is out of the depicted range (1248 nm).

FIG. 4: Encapsulation efficiency of Myrcludex B for surface-coated nanoparticles in comparison to unfunctionalized nanoparticles (n≥5). All nanoparticles show comparable values.

FIG. 5: A) shows the size of a surface-coated nanoparticle functionalized with the linear CPP, and B) shows the size of a surface-coated nanoparticle functionalized with the cyclic CPP. The size of both nanoparticles (130 to 150 nm) matches the size determined by the zetasizer measurements.

FIG. 6: Comparison of the nanoparticle size and the polydispersity index (PDI) prior to and after freeze-drying using sucrose as the lyoprotectant at various concentrations (means±SD; n≥5).

FIG. 7: Stability of the linear CPP and the cyclic CPP in simulated gastric and intestinal fluid over one hour as detected by HPLC/MS.

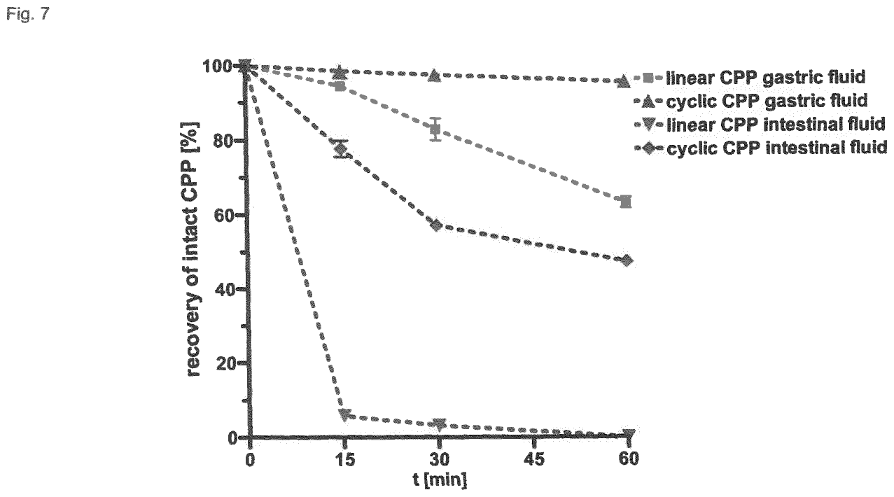

FIG. 8: Liver enrichment of I-131 radiolabeled Myrcludex B, 5 hours after the oral application (n≥3).

FIG. 9: Release of nanoparticles as a function of time for the surface-coated nanoparticles functionalized with the cyclic CPP as well as for the unfunctionalized nanoparticles.

FIG. 10: Release of nanoparticles as a function of time for the surface-coated nanoparticles functionalized with the cyclic CPP as well as for the PEGylated surface-coated nanoparticles functionalized with the cyclic CPP. Regarding the PEGylation, the release is also shown for the case in which the drug Liraglutide is encapsulated by the nanoparticles.

FIG. 11: Fluorescence microscopic evaluation of mucoadhesion and Liraglutide uptake of four different nanoparticle formulations on CaCo2 cells. “Control A” represents unfunctionalized nanoparticles, “Control B” represents PEGylated nanoparticles, “Vesicle C” represents surface-coated nanoparticles functionalized with the cyclic CPP, and “Vesicle D” represents PEGylated surface-coated nanoparticles functionalized with the cyclic CPP.

The present invention will be further illustrated by the following examples without being limited thereto.

### EXAMPLES

### Materials and Methods

Materials

Maleimide-modified polylactide (PLA) chains with an average molecular weight of 5,000 g/mol were applied from Sigma Aldrich (Steinheim, Germany) and Amicon®Ultra-4 Centrifugal filters were obtained from Merck Millipore (Tullagreen, Ireland), while Filtropur S 0.2 sterile filters were purchased from Sarstedt (Nümbrecht, Germany). Dulbecco's phosphate buffered saline was applied from gibco® by life technologies™ (Paisley, UK), Tween 85 from Sigma Aldrich (Steinheim, Germany) and radioiodine I-131 was purchased from Perkin Elmer® (Boston, USA), while Triton™ X-100, cholesterol, chloroform, methanol and all other solvents were obtained from Sigma Aldrich (Taufkirchen, Germany).

The cyclic cysteine R9 derivative (SEQ ID NO: 8; RRRRRRRRRC) as a cyclic cell-penetrating peptide (inventive Example) and linear penetratin terminated with a cysteine moiety (SEQ ID NO: 10; RQIKIWFQNRRMKWKKC) as a linear cell-penetrating peptide (comparative Example) were produced by solid-phase synthesis as known in the art using the fluorenylmethoxycarbonyl/t-butyl (Fmoc/tBu) chemistry on an Applied Biosystems 433A peptide synthesizer. Tyrosine-modified Myrcludex B (hereinafter also referred to as “Myrcludex B” only) as the peptide drug to be encapsulated was synthesized similarly. The additional tyrosine moiety was introduced for radio-labeling by iodination for animal trials.
Radiolabeling of the Tyrosine-Modified Myrcludex B

To 25 μL of the peptide drug Myrcludex B modified with a tyrosine moiety (1 mM stock solution in water/phosphate buffer pH 7.4), the required amount of radioactive iodine-131 (¹³¹l) was added. The radiolabeling was performed using the chloramine T method according to a method known in the art. The reaction mixture was purified by semi-preparative HPLC as known in the art. Afterwards, the purity of the radiolabeling was determined by radio-HPLC (Agilent 1100 series) using a Chromolith® Performance RP-18e 100-3 mm column applying a linear gradient of 0.1% TFA in water (eluent A) to 0.1% TFA in acetonitrile (eluent B) within 5 minutes; flow rate 2 mL/min; UV absorbance λ=214 nm; γ-detection.
Preparation of Surface-Coated Nanoparticles

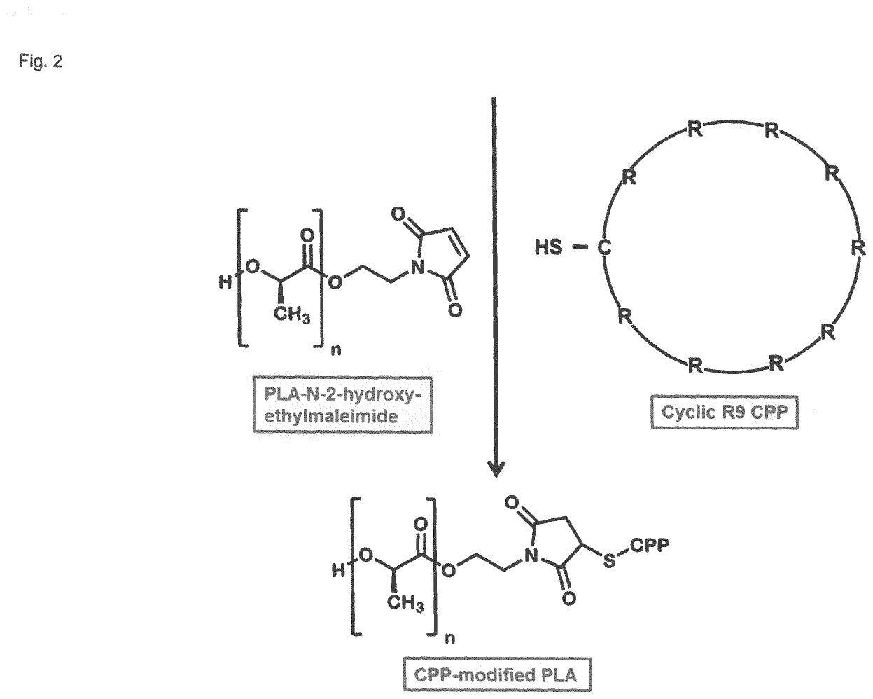

Surface-coated nanoparticles were prepared by a modified double emulsion technique as known in the art. First, the peptide drug Myrcludex B and the maleimide-modified polylactide were dissolved in 1 mL acetone followed by sonication for 5 min. Afterwards, the resulting mixture was added dropwise into 2 mL of a Tween 85 aqueous solution (0.5% v/v) while constant and fast stirring. The required amount of the respective cell-penetrating peptide was dissolved in another 2 mL of an aqueous solution of Tween 85 (0.5% v/v) and finally added to the obtained mixture. The functionalization principle for the cyclic cysteine R9 derivative can be taken from FIG. 2. Before characterization, the surface-coated nanoparticles were stirred overnight in order to evaporate the organic solvent.
Characterization of Surface-Coated Nanoparticles

The particle size, polydispersity index (PDI) and zeta potential of the surface-coated nanoparticles were determined at room temperature using the automatic mode of a Zetasizer Nano ZS from Malvern™ (Malvern Instruments Ltd., Worcestershire, UK). Size and PDI were measured after dilution to a PLA concentration of 0.10 mg/mL with a 10 mM phosphate buffer pH 7.4, while the zeta potential was determined after dilution to a PLA concentration of 0.20 mg/mL by a 50 mM phosphate buffer pH 7.4. The default settings of the automatic mode of the Zetasizer Nano ZS were the following: number of measurements=3; run duration=10 s; number of runs=10 to 50; equilibration time=120 s; refractive index solvent 1.330; refractive index polystyrene cuvette 1.590; viscosity=0.8872 mPa s; temperature=25° C.; dielectric constant=78.5 F/m; backscattering mode (173°); automatic voltage selection; Smoluchowski equation.
Encapsulation Efficiency of Myrcludex B

The encapsulation efficiency of Myrcludex B was determined by reversed phase HPLC (Agilent 1100 Series) using a C18 column (Chromolith® Performance RP-18e, 100-3 mm) applying a linear gradient of 0.1% TFA in water (eluent A) to 0.1% TFA in acetonitrile (eluent B) within 5 minutes as known in the art. After preparation, two samples of the surface-coated nanoparticles (1 mL each) were sterile filtrated by Filtropur S 0.2 sterile filters. The first sample was used to calculate the 100%-value after dissolving the surface-coated nanoparticles by acetonitrile (1:1 v/v), while the other sample was purified from not entrapped Myrcludex B by centrifugation for 30 min in Amicon®Ultra-4 Centrifugal filters. After dissolution by acetonitrile (1:10 v/v), the sample was injected in the HPLC in order to calculate the X-% value of entrapped Myrcludex B by the following equation under consideration of different sample volumes:

$$E ( \% ) = \frac{[ AUC ] Myrcludex B part 2}{[ AUC ] Myrcludex B part 1}$$

In the above equation, “[AUC] Myrcludex B part 2” is the concentration of Myrcludex B of the surface-coated nanoparticles after purification and “[AUC] Myrcludex B part 1” is the concentration of Myrcludex B in the unpurified suspension.
Characteristics of Surface-Coated Nanoparticles Using Various Surfactants

Various surfactants covering the whole HLB range have been intensively examined for the preparation of the surface-coated nanoparticles, above all polyvinyl alcohol (PVA) in several concentrations. All surface-coated nanoparticles were prepared and characterized as described above.
Cryo-EM Micrographs

The surface-coated nanoparticles were concentrated by Amicon®Ultra-4 Centrifugal filters to obtain a PLA concentration of 5 mg/mL. Quantifoil grids (2/2) were glow discharged for 20 s in a H₂ and O₂ gas mixture. 3 μL samples were applied to the grid and blotted at 4° C. and 100% humidity for 8 to 10 s in a FEI Vitrobot™. The grids were observed in a Krios™ microscope operated at 200 kV and liquid nitrogen temperature. The micrographs of the nanoparticles were taken at 64,000×magnification.
Long Term Storage Stability
Freeze-Drying Using Sucrose at Different Molar Ratios

The surface-coated nanoparticles were freeze-dried with a main drying carried out at −20° C. for 2 days, followed by a secondary drying at 0° C. for at least 6 hours in a Delta 1 to 20 KD from Martin Christ (Osterode, Germany). Sucrose in a range of 100 to 1000 mM was used as a lyoprotectant as described in the art. Briefly, the surface-coated nanoparticles were prepared as described above, and the required amount of sucrose was added to portioned 50 μL aliquots. Afterwards, the aliquots were shock-frozen in liquid nitrogen and freeze-dried. In order to assess the quality of the freeze-dried products, the surface-coated nanoparticles were rehydrated with 50 μL phosphate buffer (10 mM; pH 7.4), and the size and PDI were determined.
Residual Moisture

In order to verify the quality of the freeze-dried product, the residual moisture of all surface-coated nanoparticles was determined by a moisture meter (Kern & Sohn GmbH, Balingen, Germany) using 25 mg of the freeze-dried surface-coated nanoparticles by heating up to 120° C. in 90 seconds.
Stability Assays and Recovery of Intact Myrcludex B
Stability of Surface-Coated Nanoparticles and Recovery of Intact Myrcludex B

Surface-coated nanoparticles were diluted 1:1 (v/v) with either simulated gastric fluid or simulated intestinal fluid and incubated at 37° C. under constant shaking as described in the art. After 0, 15, 30 and 60 min, samples were analyzed by a zetasizer to obtain the size and PDI of the particles as well as by HPLC in order to detect the recovery of intact Myrcludex B. In the zetasizer analysis, for each time point, 50 μL of the sample with the surface-coated nanoparticles was diluted with 950 μL of 10 mM phosphate buffer pH 7.4. For HPLC analysis, the sample with the surface-coated nanoparticles was diluted 1:2 (v/v) with acetonitrile.
Stability of Cell-Penetrating Peptides in Simulated Gastric and Intestinal Fluid

Both CPPs (1 mg/mL in water) were diluted 1:1 (v/v) with either simulated gastric fluid or simulated intestinal fluid and incubated at 37° C. under constant shaking as described in the art. After 0, 15, 30 and 60 min, the samples were analyzed by HPLC/MS in order to detect the recovery of intact CPP and were compared in relation with the initial solution.
Cytotoxicity Assays
Cell Cultivation

CaCo2 cells were cultivated in DMEM (Gibco) supplemented with 10% fetal calf serum, 1 mM sodium pyruvate, GlutaMAX® (4 mM L-alanyl-glutamine) and 1% non-essential amino acids. The cells were cultured at 37° C. in an atmosphere of 95% air and 5% CO₂. Subcultures were taken when cells reached 80% confluence.
Cytotoxicity Assay

CaCo2 cells were seeded into 96 well plates (greiner bio-one) and grown for 14 days after the formation of a monolayer. The medium was changed every 2 days. The surface-coated nanoparticles were added in appropriate concentrations and incubated for 3 hours. Subsequently, the medium was replaced by growth medium supplemented with 10% Alamar Blue® (BIO-RAD antibodies) and cells incubated for 8 hours. Fluorescence was measured on an Infinite Tecan Platereader at a wavelength of 590 nm with an excitation wavelength of 560 nm. The cell viability was normalized to values of wells containing untreated cells as a positive control and wells containing no cells as a negative control.
Animal Trials
Proof of Concept Study

The animal study was performed according to local authorities using male Wistar rats with a body weight of about 200 to 250 g. In this study, the tyrosine-modified analogue of the lipopeptide Myrcludex B was labeled with ¹³¹l and encapsulated by the surface-coated nanoparticles as described above. 3 hours after oral administration, the organ distribution was measured by direct counting of the radiolabeled sample. Briefly, three groups (n≥3) of Wistar rats were formed.

Prior to 12 hours of the experiment, the rats were kept without food, but with free access to water. Oral application of the surface-coated nanoparticles and the free peptide drug took place by gavage. Each rat of group 1 obtained a dose corresponding to 0.5 Mega Becquerel (MBq) of the labeled free peptide drug (negative control), while each rat of group 2 obtained a dose corresponding to 0.5 MBq of the surface-coated nanoparticle functionalized with the linear CPP, and each rat of group 3 obtained a dose corresponding to 0.5 MBq of the surface-coated nanoparticle functionalized with the cyclic CPP. The rats were sacrificed 3 hours after oral application, the organ tissues were removed, weighed and the radioactivity was measured using a Berthold LB 951 G counter in comparison with standards. Due to the specific accumulation of Myrcludex B in the liver, the liver-associated activity was related to the total injected dose (ID) and expressed as a percentage of the total injected dose per gram of tissue (% ID/g).
Statistical Analyses

The statistical data were processed using the Prism® software (GraphPad Software, San Diego, Calif., USA) and presented as mean±standard deviation of the mean (SD). The different groups of the animal trial were compared by one-way ANOVA test using the Prism® software and considered significant at *p<0.05, **p<0.01 and ***p<0.001.
Results and Discussion
Characterization of Surface-Coated Nanoparticles

Various surfactants covering the whole HLB range for the preparation of the surface-coated nanoparticles have been examined, which can be taken from Table A.

TABLE A
HLB values of various surfactants
tested for nanoparticle preparation

| surfactant | HLB value |
|---|---|
| Span 85 | 1.8 |
| Span 20 | 8.6 |
| Tween 85 | 11 |
| Cremophor ® EL | 12 |
| polyvinyl alcohol | 18 |
| Kolliphor 188 | 29 |

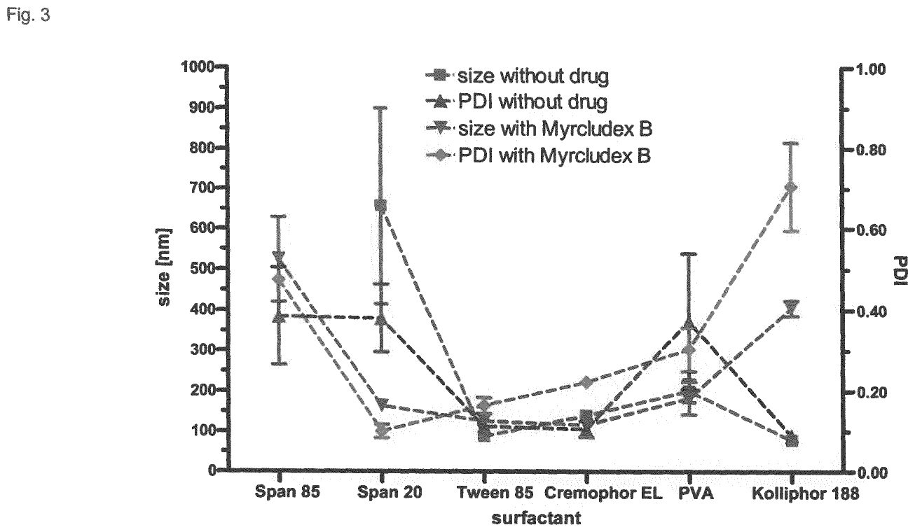

Surfactants with HLB values in a range of from 10 to 14 such as Tween 85 and Cremophor® EL showed the best results for the surface-coated nanoparticles with respect to their size and their PDI (FIG. 3). Interestingly, the combination of polylactide and polyvinyl alcohol (PVA), one popular combination for nanoparticle preparation, could not provide similar results.

Surface-coated nanoparticles prepared with Tween 85 as a surfactant showed comparable and reasonable values regarding size and PDI as determined by a zetasizer, which can be taken from Table B. The average size of the surface-coated nanoparticles either functionalized with the linear CPP or with the cyclic CPP was in a range of from 120 to 160 nm, while the PDI showed a slight increase for the surface-coated nanoparticles functionalized with the cyclic cell-penetrating peptide. Using polyvinyl alcohol as a surfactant, an average size of 200 to 250 nm was obtained for the surface-coated nanoparticles. Accordingly, the use of Tween 85 as a surfactant could provide surface-coated nanoparticles with a significant smaller average size. This demonstrates the great influence of the surfactant for the characteristics of the surface-coated nanoparticles. The zetapotential of the surface-coated nanoparticles showed a strong increase compared to the non-coated nanoparticles due to the positively charged amino acids forming the CPPs, which indicates the successful functionalization.

TABLE B
Nanoparticle characterization by a zetasizer

| | | | zetapotential |
|---|---|---|---|
| formulation | size [nm] | PDI | [mV] |
| unfunctionalized | 125.10 ± 3.92 | 0.164 ± 0.016 | −15.63 ± 5.70 |
| linear CPP | 128.75 ± 4.61 | 0.189 ± 0.025 | −2.50 ± 1.02 |
| cyclic CPP | 159.70 ± 10.13 | 0.169 ± 0.012 | −3.20 ± 1.22 |

Encapsulation Efficiency of Myrcludex B

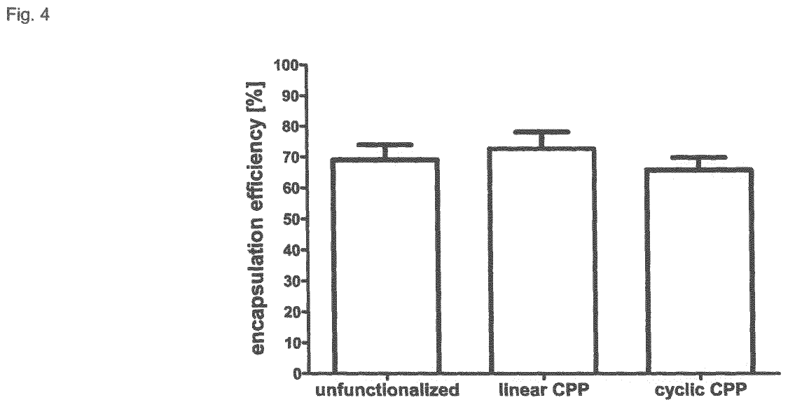

The surface-coated nanoparticles comprising Myrcludex B showed an encapsulation efficiency of 72.69±12.13% for the linear CPP and 65.73±8.99% for the cyclic CPP. These values are comparable to the value determined for the unfunctionalized nanoparticles (69.10±11.02%; FIG. 4). In comparison to encapsulation efficiencies obtained for other drugs such as Doxorubicin (40 to 70%) and Insulin (40 to 70%), the surface-coated nanoparticles provided similar results.
Cryo-TEM

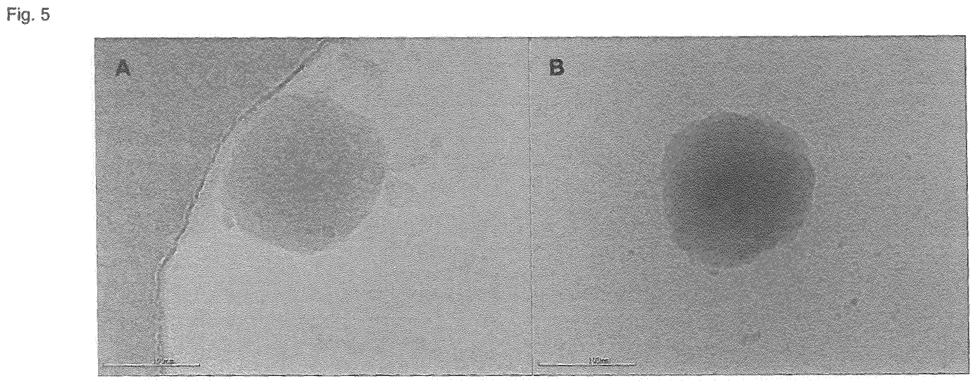

The cryo-electron micrographs (FIG. 5) show the size and structure of the surface-coated nanoparticles functionalized with the linear CPP and with the cyclic CPP. The size of both nanoparticles (130 to 150 nm) matches the size determined by the zetasizer measurements.
Freeze-Drying

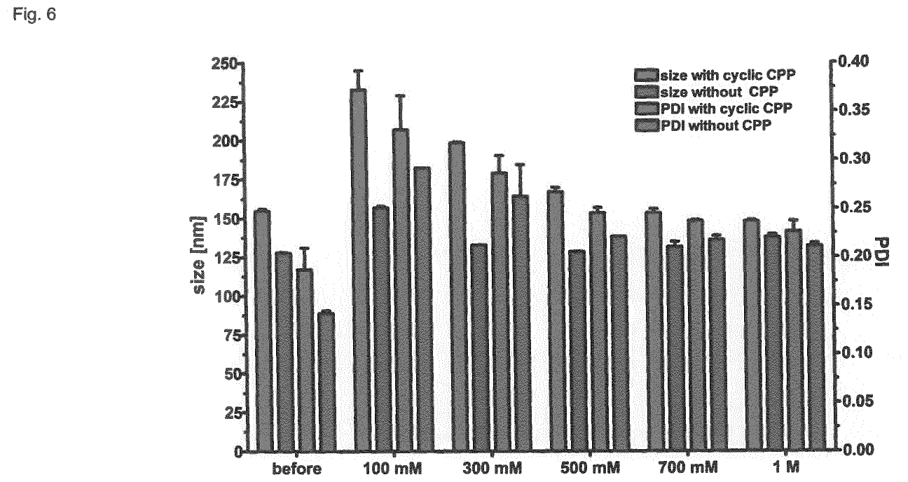

The freeze-drying of the surface-coated nanoparticles functionalized with the cyclic CPP and the freeze-drying of the unfunctionalized nanoparticles containing sucrose in different molar ratios (100 mM to 1000 mM) as a lyoprotectant resulted in a comparable size and PDI for certain molar ratios of sucrose when compared to the data measured prior to the freeze-drying process (FIG. 6). However, regarding the surface-coated nanoparticles functionalized with the linear CPP, a significant increase in size and PDI after freeze-drying could be observed. With regard to the unfunctionalized nanoparticles, the minimal concentration of sucrose should be at least 300 mM, while for the surface-coated nanoparticles functionalized with the cyclic CPP, a minimal concentration of 500 mM is required. For both surface-coated, i.e. functionalized, and unfunctionalized nanoparticles, a further increase in the concentration of the lyoprotectant did not provide significantly better results regarding the size and PDI of the nanoparticles.
Stability Assay of Cell-Penetrating Peptides and Recovery of Intact Myrcludex B

The stability assay of the linear CPP and the cyclic CPP showed a great difference in the stability in simulated gastric and intestinal fluid. Regarding the linear CPP, a moderate loss in simulated gastric fluid could be detected, while no stability in intestinal fluid could be observed (only 5.79% of intact linear CPP after 15 min). In contrast, the cyclic CPP showed a high stability in gastric fluid. Furthermore, in comparison with the linear CPP, only a moderate loss could be observed in intestinal fluid (still 47.27% of intact cyclic CPP after one hour). For this reason, functionalizing the modified polylactide chains forming the nanoparticle with cyclic CPPs may be regarded as having more potential for oral drug delivery.

The stability assay of the surface-coated nanoparticles functionalized with the cyclic CPP showed only a small loss of the peptide drug Myrcludex B in simulated gastric fluid, while no significant loss thereof could be observed in simulated intestinal fluid over one hour. This assay demonstrated that surface-coated nanoparticles functionalized with the cyclic CPP were stable in both gastric and intestinal fluid, and thus, represent a useful tool for the oral delivery of drugs sensitive to an enzymatic degradation.

TABLE C
Recovery of intact Myrcludex B in gastric
and intestinal fluid (n ≥ 5)

| | recovery of intact | recovery of intact |
|---|---|---|
| | Myrcludex B in gastric | Myrcludex B in |
| time [min] | fluid [%] | intestinal fluid [%] |
| 0 | 100 ± 0.00 | 100 ± 0.00 |
| 15 | 98.03 ± 0.23 | 98.23 ± 0.17 |
| 30 | 95.14 ± 0.57 | 96.20 ± 1.87 |
| 60 | 90.03 ± 1.13 | 95.56 ± 1.03 |

Cytotoxicity Assay

Both unfunctionalized nanoparticles and surface-coated nanoparticles showed no relevant toxicity in the tested concentrations in the alamar blue® assay, which can be taken from Table D. Altogether, it could be demonstrated that the surface-coated nanoparticles are non-toxic in the tested concentrations as previously shown for PLA nanoparticles.

TABLE D
Cell viability (n ≥ 5) of unfunctionalized
and surface-coated nanoparticles

| | cell | cell | cell |
|---|---|---|---|
| PLA concentration | viability [%] | viability [%] | viability [%] |
| [mg/mL] | unfunctionalized | linear CPP | cyclic CPP |
| 1.25 | 97.83 ± 0.39 | 99.12 ± 0.12 | 99.51 ± 0.47 |
| 0.625 | 98.97 ± 0.97 | 98.80 ± 0.46 | 99.23 ± 1.67 |
| 0.313 | 97.19 ± 1.97 | 99.16 ± 0.78 | 99.22 ± 1.50 |

Animal Trials

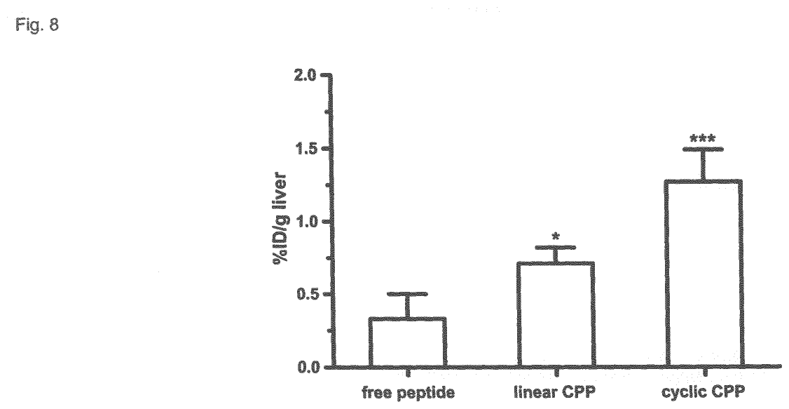

The animal studies showed a significant, i.e. about 4-fold increase in the enrichment of the peptide drug Myrcludex B in the liver tissue (FIG. 8) for the surface-coated nanoparticles functionalized with the cyclic CPP (1.27%ID/g), when compared to the labeled free peptide drug (0.33% ID/g). Regarding the surface-coated nanoparticles functionalized with the linear CPP, only a moderate increase in the liver uptake (0.71%ID/g) could be obtained. Recently published data showed a comparable increase (1.14%ID/g) in the uptake of Myrcludex B using liposomes containing tetra-ether lipids.

The above results highlight the benefit in the oral availability of Myrcludex B by the use of the surface-coated nanoparticles functionalized with cell-penetrating peptides. Nevertheless, a significant difference between the linear and the cyclic CPP functionalization could be demonstrated, which is probably due to the minor stability of the linear CPP in the gastric and intestinal fluid.
Mucoadhesivity of Surface-Coated Nanoparticles Loaded with the Drug Liraglutide

Further to the experiments carried out with the drug Myrcludex B, surface-coated nanoparticles such as prepared above, i.e. nanoparticles based on polylactide chains functionalized with the cyclic cysteine R9 derivative (SEQ ID NO: 8; RRRRRRRRRC), were loaded with the drug Liraglutide, and investigated in terms of their mucoadhesivity. In addition, nanoparticles having a PEG linker between the polylactide chains and the cyclopeptides were prepared and loaded with the drug Liraglutide to be investigated in terms of their mucoadhesivity. Herein, the bifunctional PEG linker had eight polyethylene glycol moieties.

Liraglutide was commercially obtained as an injection solution for subcutaneous application (Victoza®, 6 mg/mL, Novo Nordisk Pharma GmbH), and was separated using preparative HPLC (Reprosil Pur 120 C18-AQ, 5 μm (250×25mm), 35-70% acetonitrile+0.1% TFA, 25 min). The isolated drug was analzyed using mass spectrometry, and then used as an agent for being encapsulated with the nanoparticles.

Besides the surface-coated nanoparticles with or without PEGylation, unfunctionalized nanoparticles were used as a reference system. These unfunctionalized nanoparticles only comprised the polylactide chains as such.

Further to the nanoparticles loaded with the drug Liraglutide, unloaded nanoparticles were also investigated in terms of their mucoadhesivity.

In brief, as could be experimentally observed, the surface-coated nanoparticles having a PEG linker showed the highest mucoadhesivity while the unfunctionalized nanoparticles showed the lowest mucoadhesivity among the systems studied.
Method

For determining the mucoadhesivity of the surface-coated nanoparticles and the unfunctionalized nanoparticles on porcine intestine, a particle retention assay was performed. The experimental set-up was adopted from Preisig et al. (Int. J. Pharm. 2015, 487, 157-166). In a closed cycle with a total volume of 25 mL water at 37° C., a flow of 5 mL/min was applied. Prior to the experiment, the fixed tissue was equilibrated for 10 min and a blank sample was taken. Then, the respective nanoparticle suspension was applied to the horizontally orientated chamber and was incubated for 10 min, before the chamber was raised to a 45° angle and the flow was started. After 5, 15, 30, 60 and 120 min, samples of 200 μL were taken. 50 μL of each sample were diluted with 950 μL PBS and the derived count rate was measured by the Zetasizer Nano ZS from Malvern™ (Malvern Instruments Ltd., Worcestershire, UK) in technical triplicates. The amount of nanoparticles released from the mucosa was calculated from the ratio of the derived count rate at a given time point to the rate at time point zero (blank).
Results

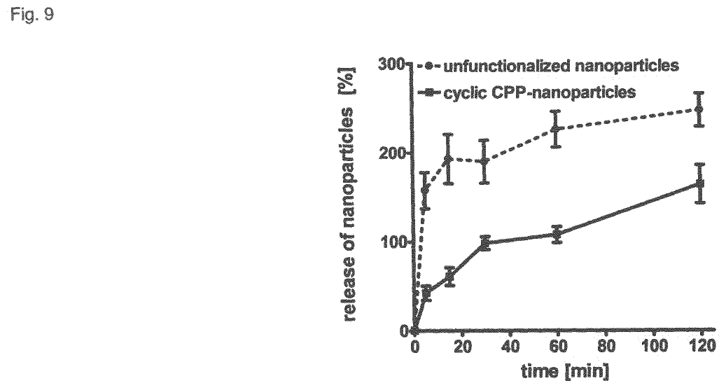

The above-described particle retention assay showed highly improved particle retention on the porcine intestine mucosa tissue for the surface-coated nanoparticles functionalized with the cyclic CPP in comparison to the unfunctionalized nanoparticles (FIG. 9). These findings can be attributed to the highly positive charge of the cyclic CPP because mucoadhesive polymers such as chitosan also exhibit a highly positive charge. The results obtained clearly demonstrate that the surface-coated nanoparticles functionalized with the cyclic CPP could also significantly enhance the retention on porcine colonic mucosa as previously described for chitosan particles by Preisig et al.

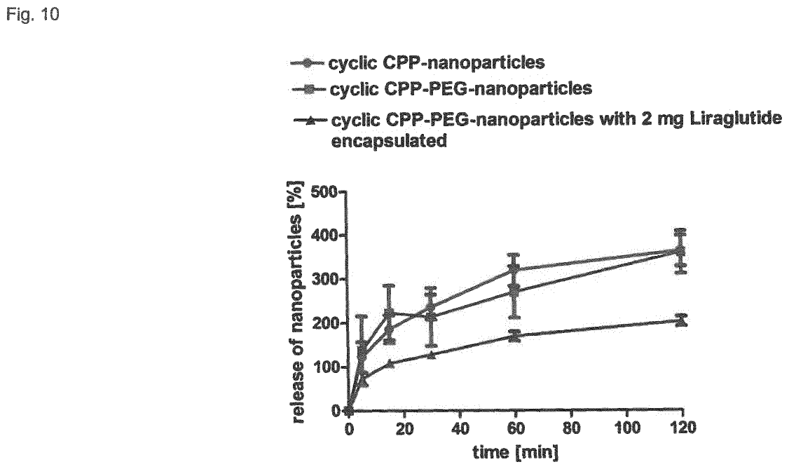

In addition, the above particle retention assay showed that the mucosa retention could be even further improved in case of PEGylated surface-coated nanoparticles functionalized with the cyclic CPP (FIG. 10).
Fluorescence Microscopic Evaluation of Mucoadhesion and Liraglutide Uptake of Four Different Nanoparticle Formulations on CaCo2 Cells

Unfunctionalized nanoparticles (“Control A”), PEGylated nanoparticles (“Control B”), surface-coated nanoparticles functionalized with the cyclic CPP (“Vesicle C”), and PEGylated surface-coated nanoparticles functionalized with the cyclic CPP (“Vesicle D”) were evaluated in terms of mucoadhesion and Liraglutide uptake on CaCo2 cells.

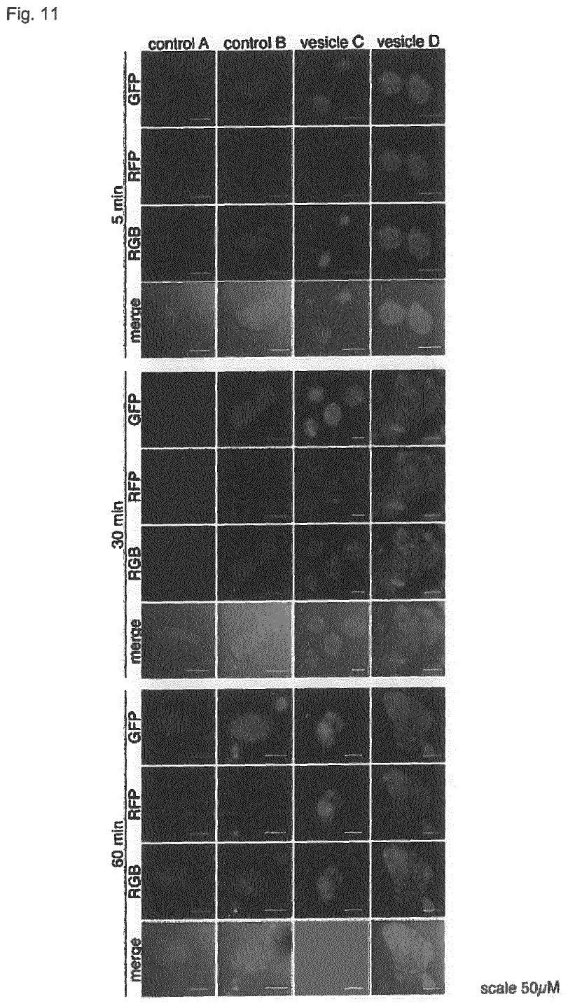

For this purpose, the drug Liraglutide was modified by an Atto-495 dye (green signal) and PLA was modified by an Atto-610 dye (red signal) to conduct fluorescence microscopic studies (FIG. 11).

Synthesis of the Liraglutide-Atto-495-Conjugate and the PLA-Atto-610-Conjugate

The Atto-495 dye was purchased as NHS-ester and coupled to Liraglutide via a threefold excess of Liraglutide. In this case, Liraglutide was dissolved in 10 mM phosphate buffer (pH 8.3) and the Atto-495 dye in an appropriate amount of dimethyl formamide. Coupling of the PLA-Atto-610-conjugate was performed in an analogous manner. Purification was achieved by preparative HPLC. The synthesis of the conjugates yielded the desired products in sufficient quantities for the following studies. Purity of all conjugates was determined by LC/MS on an Exactive mass spectrometer.
Results

As can be taken from FIG. 11, surface modification of the nanoparticles by the cyclic CPP (“vesicle C”) highly increased the mucoadhesion and also the Liraglutide uptake on CaCo2 cells.

Furthermore, it could be demonstrated that PEGylation of the cyclic CPP-nanoparticle surface also increased mucoadhesion and Liraglutide uptake over an incubation period of 60 minutes (“vesicle D”).

1. A surface-coated nanoparticle, comprising:
a nanoparticle comprising biodegradable polymer chains terminated with a functional group for being functionalized with a cyclopeptide, and cyclopeptides covalently attached to the biodegradable polymer chains via the functional group thereof, thereby coating the surface of the nanoparticle.

2. The surface-coated nanoparticle according to claim 1, wherein the biodegradable polymer is polylactide.

3. The surface-coated nanoparticle according to claim 1, wherein the degree of functionalization is between 0.1 and 10%.

4. The surface-coated nanoparticle according to claim 1, wherein the biodegradable polymer chains terminated with a functional group and the cyclopeptides are linked by a bifunctional linker, and the bifunctional linker is a bifunctional polyethylene glycol (PEG) linker having between 1 and 200 PEG moieties.

5. The surface-coated nanoparticle according to claim 1, wherein the biodegradable polymer chains terminated with a functional group have an average molecular weight of from 1,000 to 10,000 g/mol.

6. The surface-coated nanoparticle according to claim 1, wherein the isoelectric point of the cyclopeptides is higher than 7.

7. The surface-coated nanoparticle according to claim 1, further comprising an agent encapsulated therein.

8. The surface-coated nanoparticle according to claim 7, wherein the agent is a macromolecular agent.

9. The surface-coated nanoparticle according to claim 7, wherein the agent is selected from the group consisting of peptides, proteins, antibodies, and combinations thereof.

10. The surface-coated nanoparticle according to claim 7, wherein the surface-coated nanoparticle has an average size of from 50 to 500 nm.

11. The surface-coated nanoparticle according to claim 1, wherein the surface-coated nanoparticle is in a freeze-dried state in the presence of a lyoprotectant.

12. A capsule or a tablet comprising the surface-coated nanoparticle according to claim 11. 13-14. (canceled)

15. A method for the treatment or prevention of a disorder or disease in a patient, wherein the treatment or prevention comprises the oral administration of the surface-coated nanoparticle of claim 1 to the patient and is achieved by mucosal uptake of the surface-coated nanoparticle via the oral route.

16. A capsule comprising the surface-coated nanoparticle according to claim 7.


SEQUENCE LISTING (10 sequences)

SEQ ID NO: 1 - protein, 16 aa, Drosophila melanogaster
RQIKIWFQNRRMKWKK

SEQ ID NO: 2 - protein, 16 aa, Human immunodeficiency virus type 1
CGRKKKRRQRRRPPQC

SEQ ID NO: 3 - protein, 27 aa, Artificial sequence
  artificially designed cell penetrating peptide
GALFLGFLGAAGSTMGAWSQPKSKRKV

SEQ ID NO: 4 - protein, 9 aa, Artificial sequence
  artificially designed cell penetrating peptide
RRRRRRRRR

SEQ ID NO: 5 - protein, 18 aa, Mus musculus
  MISC_FEATURE (18)..(18): K-amide
LLIILRRRIRKQAHAHSK

SEQ ID NO: 6 - protein, 28 aa, Homo sapiens
  MISC_FEATURE (28)..(28): L-amide
GWTLNSAGYLLGKINLKALAALAKISIL

SEQ ID NO: 7 - protein, 27 aa, Human immunodeficiency virus
GALFLGFLGAAGSTMGAWSQPKSKRKV

SEQ ID NO: 8 - protein, 10 aa, Artificial sequence
  cyclic cysteine R9 derivative peptide
  MISC_FEATURE (1)..(10): cyclic peptide
RRRRRRRRRC

SEQ ID NO: 9 - protein, 47 aa, Hepatitis B virus
GQNLSTSNPLGFFPDHQLDPAFRANTANPDWDFNPNKDTWPDANKVG

SEQ ID NO: 10 - protein, 17 aa, Artificial sequence
  linear penetratin derivative
RQIKIWFQNRRMKWKKC